(19) United States
(12) Patent Application Publication
(10) Pub. No.: US 2024/0060118 A1
(43) Pub. Date: Feb. 22, 2024

(54) Quantum Dot-Peptide PNA-DNA Complexes as a Platform Reporter System for Multiplexed Detection in Cell-Free Transcription Translation-Based Biosensors

(71) Applicant: The Government of the United States of America, as represented by the Secretary of the Navy, Arlington, VA (US)

(72) Inventors: Scott A. Walper, Springfield, VA (US); Igor L. Medintz, Springfield, VA (US); Divita Mathur, Fairfax, VA (US); Sebastian A. Diaz, Silver Spring, MD (US); Meghna Thakur, Arlington, VA (US); Kimihiro Susumu, Washington, DC (US); Michael H. Stewart, Washington, DC (US)

(21) Appl. No.: 18/203,189

(22) Filed: May 30, 2023

Related U.S. Application Data
(60) Provisional application No. 63/347,296, filed on May 31, 2022.

(51) Int. Cl. C12Q 1/6818; C07K 7/08

(52) U.S. Cl. CPC C12Q 1/6818; C07K 7/08; C07K 2319/21

(57) ABSTRACT

A FRET based reporter system utilizing the fluorescent semiconductor quantum dots (QDs) and restriction enzymes expressed in cell-free system has the potential to detect multiple analytes in single reaction.

### CROSS-REFERENCE TO RELATED APPLICATIONS

This application claims the benefit of U.S. Provisional Patent Application No. 63/347,296 filed on May 31, 2022, which is incorporated herein by reference in its entirety.

### FEDERALLY-SPONSORED RESEARCH AND DEVELOPMENT

The United States Government has ownership rights in this invention. Licensing inquiries may be directed to Office of Technology Transfer, US Naval Research Laboratory, Code 1004, Washington, DC 20375, USA; +1.202.767.7230; techtran@nrl.navy.mil, referencing NC 210886.

### BACKGROUND

Point of care diagnostics fulfill a need for rapid testing in diverse situations. An efficient diagnostic test should respond to physiologically relevant fluids, which contain analytes, nucleic acids, proteins, and other biomolecular species. Inorganic sensors can struggle to operate with such solutions, with tendencies for slow responses or leakage. Moreover, an efficient sensor should have a reportable signal transduction mechanism that is sensitive and easily resolvable by the user without the requirement of expensive machines.

Cell-free systems represent an emerging and enabling technology with broad application to sensor development and deployment. Cell-free systems seek to mimic biological activities, specifically transcription and translation (TX-TL), with minimal components and function in the absence of encapsulating cellular membranes. Typically, the biological materials necessary for these processes including ribosomes, enzymes, and other biomolecules, are obtained from either cellular lysates or are recombinantly produced. As their name implies, cell lysates are generated through mechanical or chemical lysis of cell biomass which is subsequently separated into soluble (functional lysate portion) and insoluble fractions (membranous, vesicular). Recombinant systems, in contrast, are assembled from recombinantly-produced and purified cellular components, which are then carefully combined into functional in vitro reaction mixtures. While source materials are different, both lysates and recombinant systems function essentially the same, transcribing user added nucleic acids and then translating that to protein products. Reduced to this simplicity and unrestrained by biological toxicity, cell-free systems are conducive to bioproduction of a wide array of biomolecules including fluorescent proteins and enzymes which enable sensor development.

Biological sensors harness the specificity and speed of biological systems for the detection of unconventional targets such as nucleic acids and small organic molecules that are typically not detectable by many conventional chemical sensors and instrumentation. In biological sensors (biosensors), detection moieties can be highly variable and can include proteinaceous receptors, nucleic acid-based detectors, short peptides, and many others while output signals are typically reporter proteins or enzymes that catalyze a reaction producing a fluorescent or luminescent product. Here, and in many other biosensors, sensing occurs through manipulation of the bacterial transcription and translation process where the sensing element most commonly controls one of these two critical biological functions. As examples, in transcription factor-based systems a DNA binding protein which regulates transcription undergoes a conformational change in the presence of a target molecule (small molecule, environmental condition, etc.) leading to dissociation from the DNA molecule and subsequent gene transcription and translation of the reporter protein/enzyme. Other systems, in contrast, allow for active transcription of the gene encoding the reporter but inhibit ribosome binding and transcription until the analyte (small molecule, nucleic acid, etc.) is encountered.

While cell-free sensors are biosensors by definition, their lack of a living component imparts two distinct advantages compared to cell-based biosensors: first, cell-free systems are non-living and therefore do not need to proliferate (i.e., reproduce or grow and expand) to function in field-based detection and second, as a non-living system, cell-free systems can be integrated into materials and/or lyophilized for prolonged storage over a range of temperatures and conditions without significant loss of activity than can arise from conditions harmful to living cells.

To date, most cell-free sensors have relied on enzyme-based or fluorescent reporters that are amenable to integration into materials such as paper for assay development. Both systems have shown success in assay development, however, developing optically multiplexed capabilities has proven challenging as fluorescence-based reporters have well-defined excitation/emission profiles while enzymatic systems often show overlap with absorbance patterns for colorimetric products.

A need exists for new techniques to monitor cell-free reactions.

### BRIEF SUMMARY

Described herein is a multiplex-capable system that utilizes a collection of sensitive fluorescent outputs that can be excited within a narrow visible light range and which are activated by a specifically encoded enzyme generated by the cell-free system, for example in response to the addition of a specific analyte. This combination improves the overall time of sensor response and functionally serves to amplify the signal and response.

In a first embodiment, a cell-free reporter system includes a photoluminescent quantum dot reporter comprising a quantum dot (QD); a peptide-PNA strand comprising (1) a peptide nucleic acid (PNA) comprising a PNA sequence and (2) a peptide comprising a polyhisitine sequence effective to bind the peptide-PNA strand to the QD; a nucleic acid bridge strand comprising a sequence complementary the PNA sequence and configured to bind the bridge strand to the peptide-PNA via hybridization, a target sequence for a restriction endonuclease enzyme of interest, and a bridge terminal spacer; and a nucleic acid quencher strand comprising a sequence complementary to the target sequence thus binding the quencher strand to the bridge strand, a second terminal spacer, and quencher configured as a Forster resonance energy transfer (FRET) partner of the QD, such that the quencher is positioned to quench QD fluorescence bound to the bridge strand, wherein cleavage by the restriction endonuclease enzyme of interest causes separation of the quencher from the QD and an increase of QD fluorescence.

In a second embodiment, the reporter system comprises two or more different reporters, each having distinct quantum dots and target sequences corresponding to different restriction endonucleases of interest.

In another embodiment, a method of monitoring a cell-free reaction involves conducting a cell-free reaction effective to produce one or more restriction enzymes, introducing the reporter of the first embodiment, and monitoring fluorescence of the QD as an indication of reaction progress. In further aspects, the monitoring is multiplex monitoring of multiple distinct reactions running concurrently by using the reporter system of the second embodiment.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D illustrate the use of a QD reporter in cell-free extract. FIG. 1A depicts the general operation oQD reporter. The reporter functions in a cell-free protein synthesis extract such that the absence of analyte keeps the reporter “OFF” while addition of an analyte triggers the synthesis of a restriction enzyme that in turn reacts with the reporter to change the fluorescence readout of the reporter. FIG. 1B shows the molecular structure of the QD platform reporter. The reporter contains a semiconductor QD (grey) to which a DNA duplex (pink) is attached via a peptide-PNA strand (yellow). The peptide domain couples with the QD surface through poly-histidine metal affinity coordination while the PNA domain hybridizes with the DNA. On one DNA strand there is a dye-acceptor molecule (Cy3 in this case) that interacts with the QD donor via Forster resonance energy transfer (FRET). The DNA duplex also contains a restriction site (orange) that is recognizable by a known restriction enzyme such as BamHI. FIG. 1C depicts the mechanism of QD reporter activity. The reporter functionality is based on FRET between the QD donor and acceptor dye molecule. For example, shown here is a 525 nm emitting QD donor and Cy3 acceptor dye molecule. In the OFF state, the excitation of the QD at the appropriate wavelength triggers energy transfer from the QD (donor) to Cy3 (acceptor) resulting in a high Cy3 fluorescence readout. In the presence of the right restriction enzyme (in a cell-free extract or in buffer solution), the reporter is turned ON by the cleavage of the DNA domain, concomitant release of the Cy3 acceptor dye strand, and fluorescence de-quenching of the QD.

FIG. 1D shows representative fluorescence output of the QD reporter in the presence and absence of BamHI restriction enzyme. Inset: Measurement of the change in QD/Cy3 fluorescence ratio in the two states. In the ultimate embodiment of the full reporter format, the QD-based reporting element(s) would be placed in the cell-free reaction mix along with plasmids or nucleic acid sequences encoding one or more restriction enzymes. Addition of target analyte to the mix would trigger the transcription-translation of the restriction enzyme(s), which subsequently cleave the appropriate acceptor-labeled target DNA, and change the rate of QD donor−acceptor dye FRET. The presence of multiple QD reporter constructs allows for the detection of multiple analytes in a multiplexed-multicolor format.

FIGS. 2A-2F show spectral calibration of the QD-PDD reporters. FIG. 2A schematically illustrates three configurations of the reporter that were initially characterized in order to optimize S/N, namely, Linear, Zipper, and Zipper⁺ constructs. In FIG. 2B, one can see the titration of PNA-DNA-Dye (PDD) units per QD in the three configurations of the BamHI-based reporter. Linear construct showed poor FRET. Four PDD units per QD for Zipper and Zipper⁺ configurations was selected based on the data in FIG. 2B as it accesses the most dynamic changes in FRET. Estimated donor-acceptor distances (r_{DA}) determined using FRET theory are also listed. Titrations corresponding to EcoRI- and NcoI-based reporters are shown in the Appendix. Henceforth, the figures in this document show one PDD displayed per QD when in fact 4 PDDs per QD were displayed experimentally. FIG. 2C provides fluorescence spectra of the Zipper⁺ QD525-Cy3 reporter designed against BamHI enzyme (b-Zipper⁺) in the presence of varying proportion of pre-digested target DNA. FIG. 2D is a calibration curve derived from FIG. 2C. The spectral calibration of the remaining QD-PDD reporters is in the Appendix. Error bars indicate standard deviation from the mean (n=3). FIG. 2E shows kinetic measurements of QD/Cy3 ratio over time in the presence of increasing BamHI enzyme. FIG. 2F provides progress curves in enzyme time of restriction digestion with indicated amount of initial BamHI.

FIGS. 3A-3C depict multiplexing QD-PDD reporters against three restriction enzymes. As schematically illustrated in FIG. 3A, three QD-PDDs were combined, namely, e-Zipper⁺ against EcoRI, b-Zipper⁺ against BamHI, and n-Zipper against NcoI with different mixes of the three enzymes (8 units each), as shown in the table (right). e-Zipper⁺ uses 425 nm emitting QD with ATTO425 acceptor, b-Zipper⁺ uses 525 nm emitting QD and Cy3, and n-Zipper uses 625 nm emitting QD and AF647 acceptor. FIG. 3B shows the fluorescence spectra of the three reporters when combined in one solution (curve labeled “Exp”). Gaussian deconvolution of individual QD and acceptor dye components of each QD-PDD are also shown. FIG. 3C presents the change in response for each reporter relative to control sample (that contains no added restriction enzymes) in different mixes of enzymes after 1 hour (error bars represent standard deviation from the mean of n=3).

FIGS. 4A-4C illustrate QD-PDD functionality in the cell-free system. Activity of (A) b-Zipper+, (B) e-Zipper+, and (C) n-Zipper+ reporters in cell-free extract supplemented with the enzyme-expressing gene. Error bars represent standard deviation from the mean (n=3).

FIG. 5 shows multiplexed QD-PDD reporters in various combinations of restriction enzyme-expressing cell-free systems. Fold change response in each reporter relative to control sample (that contains no added genes) in different mixes of enzyme-expressing genes after 1 hour. Error bars indicate standard deviation from the mean (n=3).

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a”, “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

As used herein, the term “hairpin” refers to a nucleic acid structure configured from single-stranded DNA or RNA where two regions of the strand are complementary when read in opposite directions, thus tending to pair together, exposing a of single-stranded nucleic acid therebetween, thus forming a stem-loop structure.

Overview

Semiconductor quantum dots (QDs) are highly amenable as fluorescent nanoparticles for incorporation into biosensing and imaging applications as either reporters or parts of the signal transduction. Interfacing QDs with biological signals has been challenging due to limited bio-conjugation techniques. Here, constructs involving, peptides and peptide nucleic acid (PNA) serve to functionalize QDs with oligonucleotides for building reporter complexes. The reporters are termed QD-PDDs (QD peptide PNA-DNA-Dye).

Peptide nucleic acids (PNAs) are synthetic mimics of DNA in which the deoxyribose phosphate backbone is replaced by a pseudo-peptide polymer to which the nucleobases are linked. See Pellestor and Paulasova, 2004. PNAs can be prepared using techniques similar to those used for automated peptide synthesis. As would a conventional nucleic acid, a PNA will hybridize with complementary DNAs or RNAs with high affinity and specificity.

As described herein, a complete QD-PDD reporter includes a QD, a peptide-PNA bound thereto, a bridge strand bound to the peptide-PNA, and a dye strand bound to the bridge strand. The peptide-PNA includes a peptide comprising polyhistidine sequence for binding to a quantum dot, and a PNA sequence complementary to a corresponding sequence in the bridge strand. The bridge strand is a nucleic acid that has one end complementary to and binding to the appropriate PNA sequence, a target sequence recognized by a restriction endonuclease enzyme of interest (when in double-stranded form bound to its complement), and a terminal spacer to allow the enzyme to operate efficiently. The dye strand includes its own terminal spacer, the complement of the target sequence of the enzyme, and a fluorescent dye or quencher configured as a Forster resonance energy transfer (FRET) partner of the QD. The construct is configured to place the dye or quencher at position so that fluorescence of the QD is quenched when the construct is in its assembled state. Restriction enzyme activity releases the dye or quencher, resulting in QD fluorescence.

Aspects are detailed in “Hybrid Nucleic Acid-Quantum Dot Assemblies as Multiplexed Reporter Platforms for Cell-Free Transcription Translation-Based Biosensors,” ACS Synth. Biol. 2022, 11, 12, 4089-4102, which along with the corresponding Supporting Information is incorporated herein by reference for the purposes of teaching techniques for making and using QD-PDD reporters.

QD-peptide PNA conjugation can provide a range of orthogonal reporters activated in the presence of bacterial endonucleases or restriction enzymes. The QD-PDDs can share the same peptide PNA backbone but with unique QD-dye combinations. Within this assembly, a double-stranded DNA molecule serves as the tether between the QD and a proximal dye or quencher molecule which engages the QD in Forster resonance energy transfer (FRET), the nucleotide sequence of which is designed to serve as a target or substrate for a unique bacterial endonuclease. A valuable tool of genetic engineering, restriction enzymes are small bacterial defense proteins that are capable of recognizing palindromic DNA sequences, to which they can subsequently bind and cleave through hydrolysis of the phosphodiester backbone. In the presence of this enzyme, the tethering DNA chain of the QD-PDD assembly is cleaved releasing the quencher from the complex and allowing for measurable changes to FRET and to the emission from the QD reporter. The benefits of this system are two-fold: (1) all QDs absorb within the same narrow excitation wavelength and (2) the combination of the QD size-associated emission wavelength and endonuclease specificity for defined nucleic acid sequences enables pairing of reporter assemblies into multiplex assays capable of simultaneous detection of multiple analytes in a single reaction.

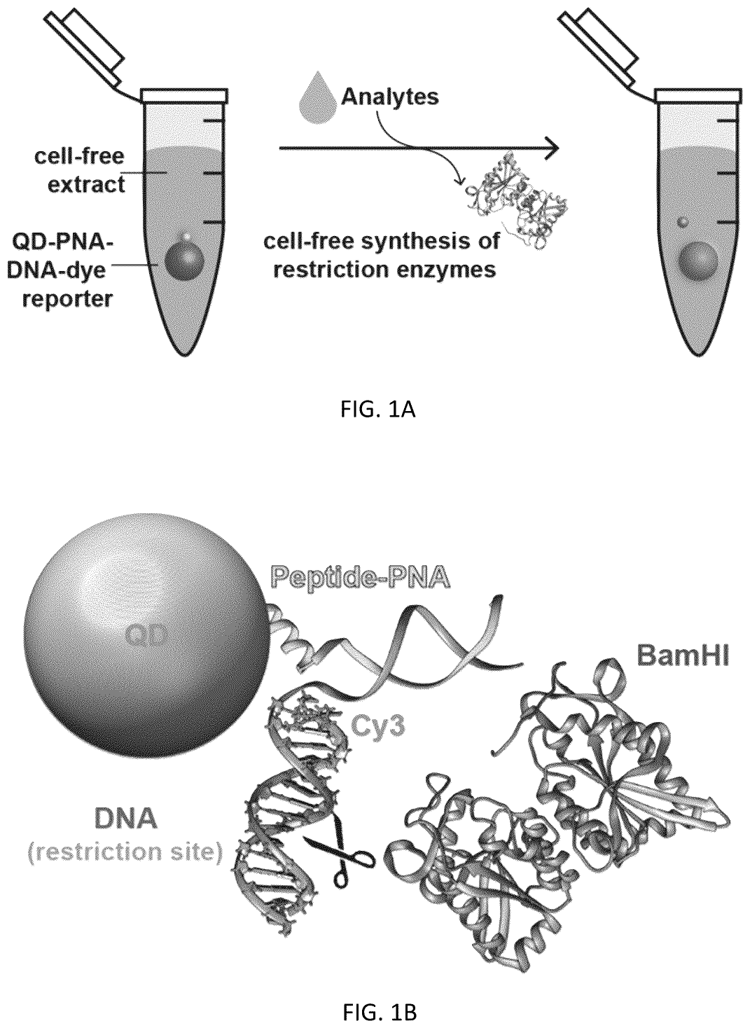

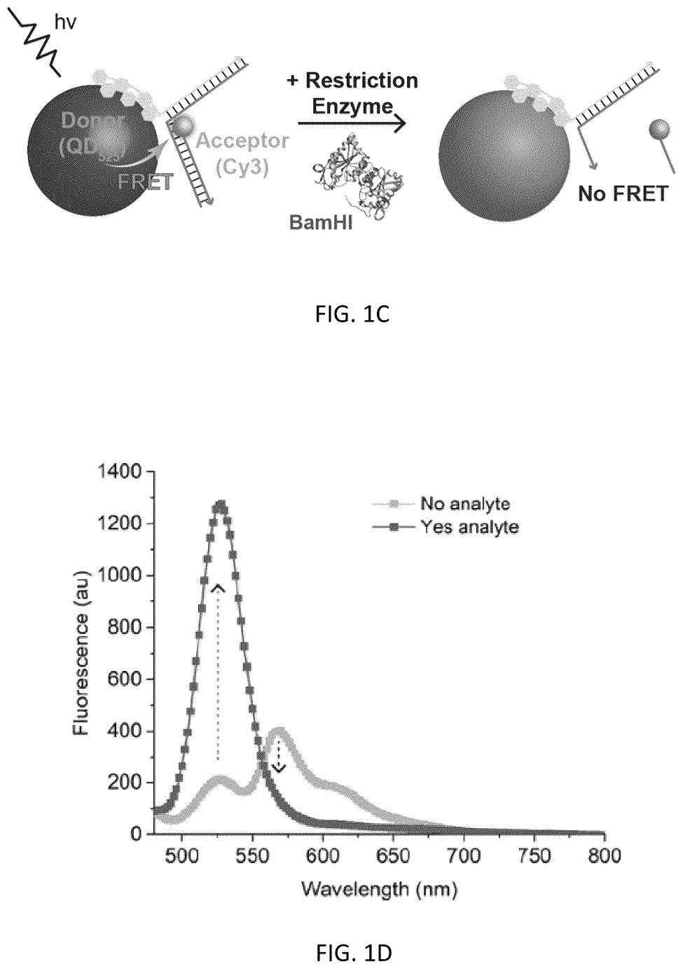

FIGS. 1A-1D show the general mode of operation of the reporter. The QD-based reporter assemblies include various differentially emitting QDs (yet can be simultaneously excited at the same wavelength) that display multiple copies of a His₆-peptide/PNA hybrid or PNA-DNA-Dye. The His₆ moiety self-assembles to the QD ZnS surface by metal affinity coordination. Attached to the PNA portion are pre-hybridized DNA sequences that also display an acceptor dye. The DNA portion contains a specific sequence that is recognized and cleaved by a unique restriction enzyme. When multiple copies of this PNA-DNA-Dye are displayed around the central QD donor, the dyes quench the QD photoluminescence and sensitize the acceptor via FRET. In the system described here, activation of a genetic circuit in the cell free reaction leads to subsequent restriction enzyme production via the cell-free TX-TL machinery. Once translated by the ribosomes within the cell-free reaction, the restriction enzyme cleaves the dsDNA portion of the PNA-DNA-Dye on the QD, which alters the rate of FRET. Having multiple such QD-PNA-DNA-Dye in the appropriately responsive reaction mix where the QDs are spectrally well-resolved and assembled with DNA recognized by different restriction enzymes allows for the simultaneous reporting of multiple analytes by the changes in the different QD (and dye) emissions.

Partnered with engineered biological sensors, including protein and nucleic acid activators such as transcription factors, toehold switches, aptamers, and the like, the QD-PNA-DNA-Dye reporters can enable development of multiplex assays with reduced complexity compared to existing systems. Unlike fluorescent protein or enzymatic reporter systems that may require multiple spectral excitation/emission monitoring wavelengths or substrate combinations, the QD-PNA-DNA-Dye reporter relies on single wavelength excitation and highly tunable output properties to convey improved sensitivities when combined with biosensor regulatory components.

### Examples

Preparation of QD-PDD Reporters

The following describes the preparation of QD-PDDs. QD used in this work were CdSe/CdS/ZnS QDs with CL4 ligands as described in commonly-owned U.S. Pat. No. 9,304,124 and Diaz et al, 2017. The peptide-PNA strand used in all QD-PDD reporters contained a hexahistidine chain at the N-terminal that self-assembles on the QD via metal-affinity coordination with surface Zn²⁺ ions (see Table S1 in the Appendix). The PNA sequence was 14-bases long and optimized for minimal off-target secondary structure formation and adequate length to achieve high temperature PNA-DNA duplex stability. PNA sequences can be varied as desired. One of the strands within the dsDNA domain of the reporter, referred to as the dye strand, was designed to be 16 nucleotides long, modified with an acceptor dye molecule on one end, and encoded with a 6-nucleotide palindromic restriction site in the middle. Taking into consideration that restriction digestion activity is affected by the steric environment around a DNA restriction site, the palindromic site on the DNA was flanked by 5 nucleotides of “spacer” sequence on both 5′ and 3′ ends to provide the recommended nominal space. The longer the spacers, the further the dye would be from the QD surface, therefore it was essential to restrict the length of the dye strand. The other strand in the dsDNA domain (bridge strand) contained two concatenated subsequences, one that was complementary to the PNA strand and the other complementary to the dye-modified ssDNA, thereby enabling the positioning of the dye and restriction site near the QD surface. Programming the QD-PDD to respond to different enzymes requires identifying the appropriate palindromic restriction site to be included in the dsDNA domain. Restriction digestion was expected to cause a double-stranded nick in the dsDNA, create a ˜8 bp free dsDNA to separate from the QD-PDD system and concomitantly alter the overall FRET and QD quenching. Assembly of the QD-PDD is rapid and simple: briefly, equimolar quantities of peptide-PNA and the two DNA strands were combined and annealed first, and subsequently added to the QD for self-assembly.

The three enzymes in this multiplexed reporting system were chosen from a pool of restriction enzymes known to serve as high-performing reporters in T7 RNA polymerase (RNAP) based cell free sensors. The fluorescent components of each QD-PDD are as follows: −425 nm emitting CdSe/CdS/ZnS QD (QD₄₂₅) and ATTO425 acceptor dye were encoded in EcoRI-reporter, 525 nm emitting QD (QD₅₂₅) and cyanine 3 (Cy3) in BamHI, and 625 nm emitting QD (QD₆₂₅) and Alexa Fluor 647 (AF647) in NcoI, as noted in Table 1 below. Each FRET pair demonstrates properties desired to design a multiplexed setup—large spectral overlap between each donor QD and acceptor dye, there exists sufficient spectral separation between the FRET pairs across the visible spectrum, and all three donor QDs can be simultaneously excited at the same UV wavelength of 350 nm.

TABLE 1
Exemplary QD-PDD constructs.

| | | | | | Restriction |
|---|---|---|---|---|---|
| | | | | | site |
| | Target | | Acceptor | | sequence |
| QD-PDD | Enzyme | QD (λₑₘ) | dye | Configuration | (5′-3′) |
| b-Linear | BamHI | 525 nm | Cy3 | Linear | GGATCC |
| b-Zipper | BamHI | 525 nm | Cy3 | Zipper | GGATCC |
| b-Zipper⁺ | BamHI | 525 nm | Cy3 | Zipper⁺ | GGATCC |
| n-Zipper | NcoI | 625 nm | Alexa | Zipper | CCATGG |
| | | | Fluor 647 | | |
| n-Zipper⁺ | NcoI | 625 nm | Alexa | Zipper⁺ | CCATGG |
| | | | Fluor 647 | | |
| e-Zipper⁺ | EcoRI | 425 nm | ATTO425 | Zipper⁺ | GAATTC |

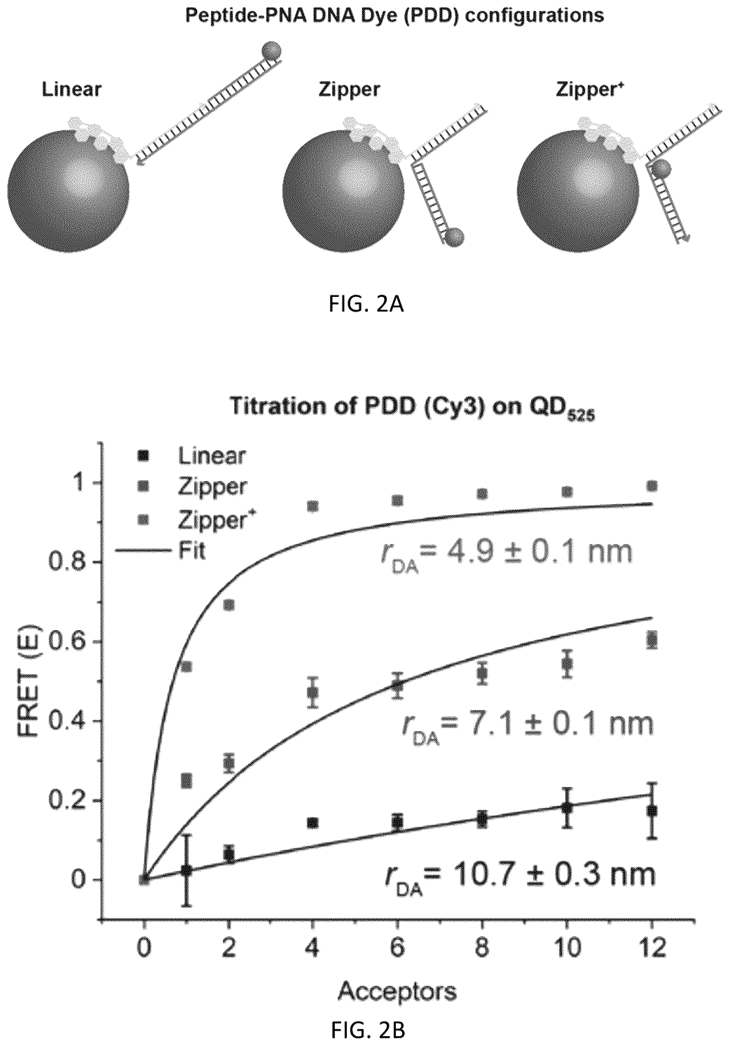

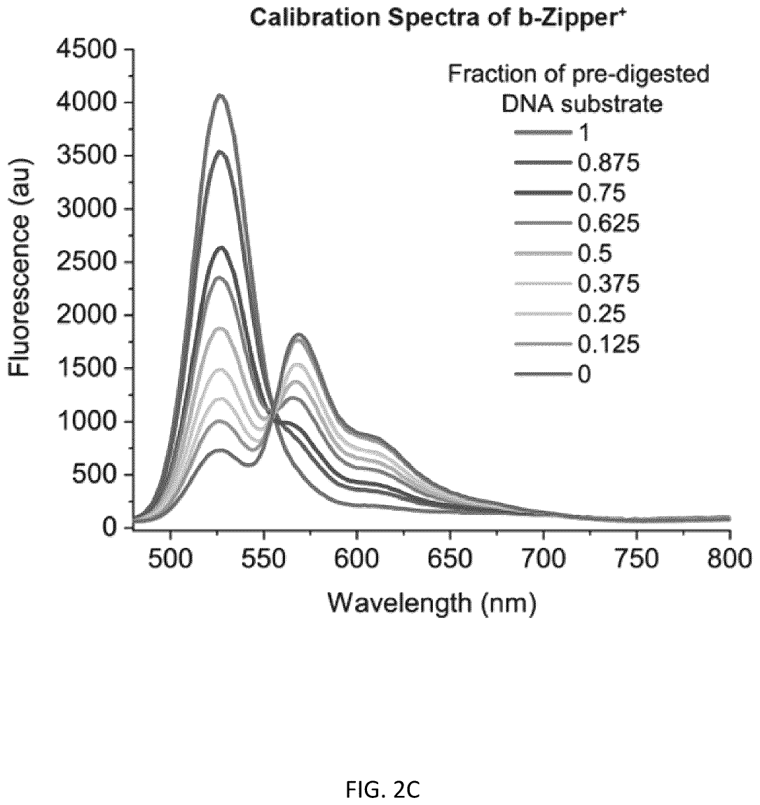

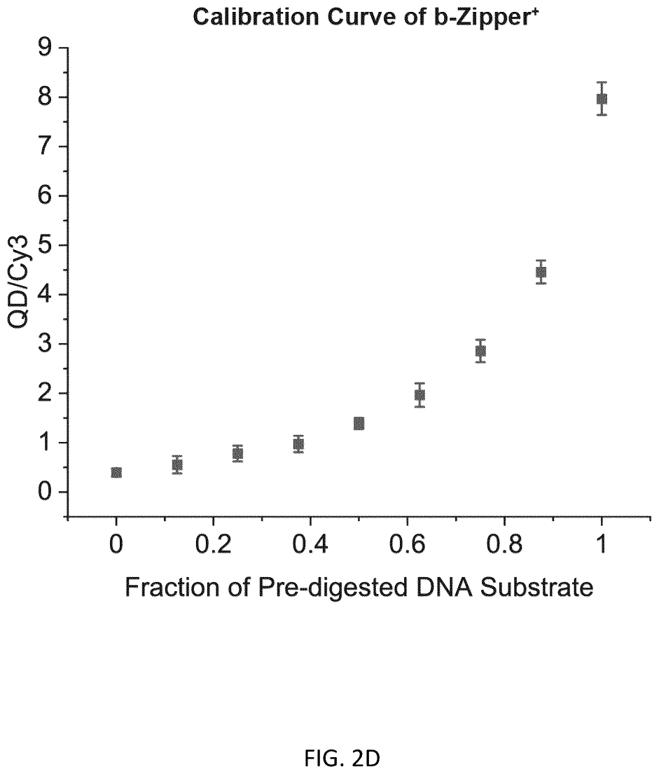

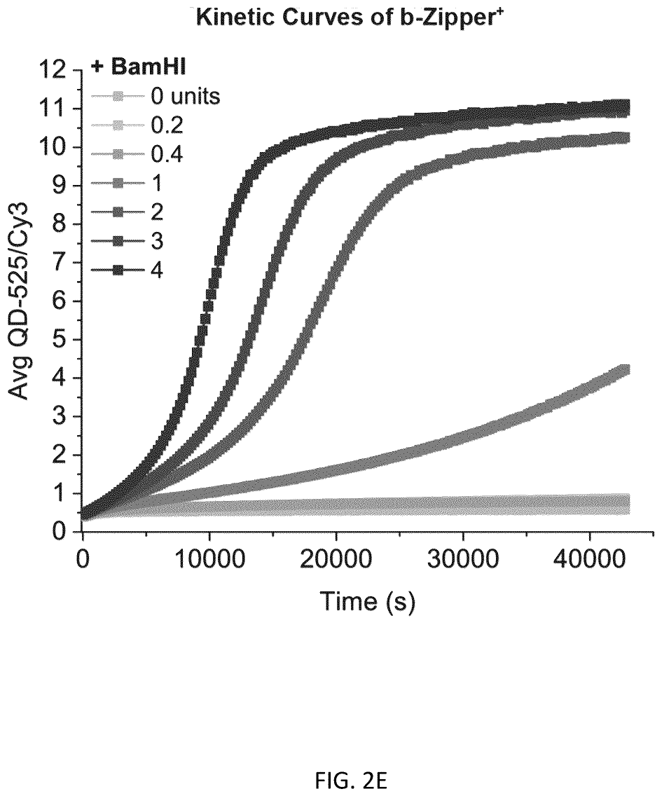

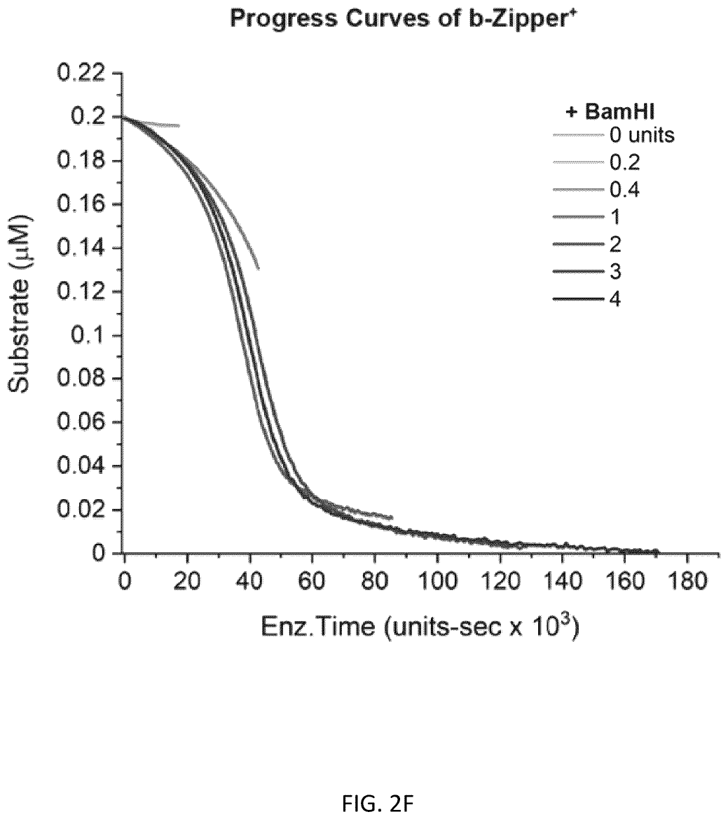

For the same QD, peptide-PNA, and restriction site, three ways of displaying the PDD portion of a reporter on QD surface were examined, as shown in FIG. 2A. A straightforward approach would be to hybridize the PNA-DNA-dye duplex in a “linear” fashion with the dye on the furthest end of the dsDNA such that restriction digestion would lead to an 8-bp dsDNA to separate from the QD-PDD. The Linear design offered the highest steric advantage for restriction enzymes (by virtue of its distance from QD surface) to access the dsDNA site and cleave but extended the acceptor dye furthest from the QD surface, thereby lowering FRET (FIG. 2B). Based on Forster theory, the calculated donor-acceptor separation r_{DA} was found to be 10.7±0.3 nm in the initial studies using 525 nm emitting QD and Cy3 acceptor-containing PDDs. Zipper configuration has a reversed orientation of the dsDNA hybridization with the PNA which significantly closes the distance between the acceptor dye and QD for improved FRET (r_{DA}7.1±0.1 nm). The Zipper configuration still afforded the placement of the dye at the DNA end that was expected to separate post restriction digestion. Finally, also tested was a modified Zipper—named Zipper⁺—where the dye was coupled to the end closest to the QD surface (thereby performing at the highest FRET). It was hypothesized that restriction digestion would create an 8-bp ssDNA segment on the QD-PDD that would separate from the system due to thermal instability (r_{DA} 4.9±0.1 nm).

QD-PDD Performance with Commercially Available Enzymes

The three configurations were tested on a series of BamHI-based reporters—b-Linear, b-Zipper, and b-Zipper⁺. To determine the FRET efficiency as a function of the number of PDDs displayed on a QD surface, different QD:PDD ratio reporters were assembled, from 0 PDDs per QD to 12 PDDs per QD. The FRET output for all three design configurations (Linear, Zipper, and Zipper⁺) showed that 4 PDDs per QD resulted in the most dynamic change of the FRET efficiency upon PDD cleavage (FIG. 2B). Thus, subsequent experiments were performed with QD-PDDs formed at 4-fold-PDD to QD ratio. In addition, the performance of the Linear configuration was poorer and, therefore, was not pursued further. Further characterization of the b-Zipper⁺ system was conducted following an approach to study enzyme-substrate kinetics on a QD surface (FIGS. 2C-F). The reporter demonstrated a large range of QD/Cy3 change against enzyme concentration. Attempts to calculate Michaelis-Menten (MM) kinetic parameters based on the experimental data, such as seen in FIGS. 2E and F, were unsuccessful. Though signal velocity correlated with enzyme concentration, deviations from the MM assumptions, most likely the free diffusion of both substrate and enzyme are not met. This has been observed in QD-based protease sensors, were enzymes are capable of undergoing a ‘hopping’ mechanism that results in deviations from MM. Similarly, b-Zipper, Zipper/Zipper⁺ reporters against NcoI, and Zipper⁺ against EcoRI were tested, shown in Appendix Figure S2. Nevertheless, quantitative units of enzymatic velocity or concentration of substrate cleaved per unit time could be collected for each. It was demonstrated that all systems are active to their desired enzyme and though they don't fully follow MM mechanism, the cleavage of the palindromic DNA and subsequent signaling is enzyme concentration dependent. The spectral overlap and FRET (Appendix Figure S2B) between 425 nm emitting QD and ATTO425 (used in e-Zipper⁺ QD-PDD) are low (Appendix Figure S1D, ˜0.6×10¹⁴ M⁻¹cm⁻¹nm⁻¹), thus the Zipper configuration for EcoRI reporter were not tested, as it would have even lower FRET.

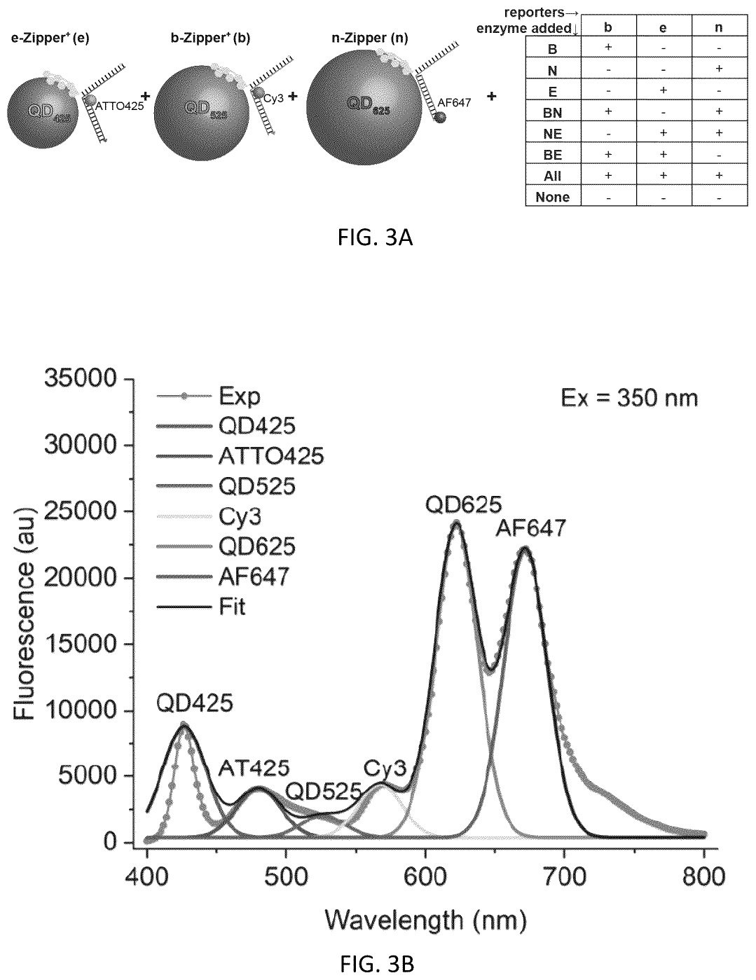

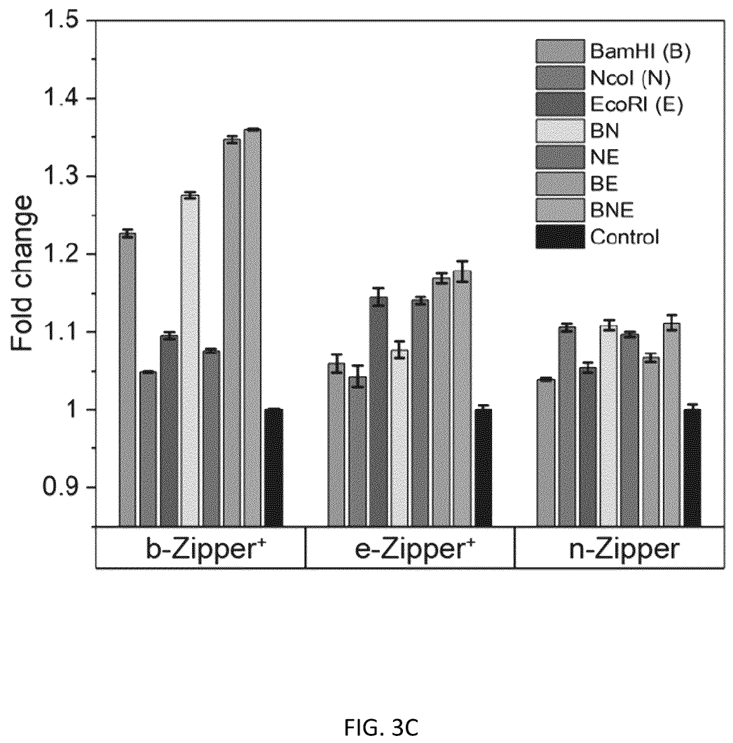

Testing with commercially purchased enzymes was realized to confirm activity, specificity, and determine if crosstalk between the reporters existed, with data provided in FIGS. 3A-3C. The Zipper⁺ configurations for BamHI and EcoRI, and the Zipper configuration for NcoI were selected post-calibration since the enzymatic activity in those reporters was relatively more predictable (Appendix Figures S2 and S3). The amount of enzyme added in the 8 different combinations was fixed at 8 units per enzyme for 2 μM total PDD (acting as substrate) per QD reporter concentration and 200 μL reaction volume, where 1 enzyme unit is defined by the vendor as the amount of enzyme that cleaves 1 μg of DNA in 1 hour at 37° C. Equimolar b-Zipper⁺, e-Zipper⁺, and n-Zipper QD-PDDs were combined to prepare a triple-reporter mix, which was subsequently divided into 8 samples to test unique cocktails of enzymatic treatments, as listed in the table of FIG. 3A. A complete steady-state spectrum of the triple-reporter mix showed distinct emission peaks corresponding to each fluorescent component in the mix—425/525/625 nm emitting QDs as well as their acceptor dyes ATTO425/Cy3/AF647 (FIG. 3B). Moreover, a Gaussian fit of the spectrum deconvolved the spectrum into individual components and nominal crosstalk between each emission spectrum can be observed, making the system useful in the development of point-of-care sensors. The performance of the triple-reporter mix is represented as fold change, which is defined as the ratio of QD/acceptor emission in a particular sample divided by the QD/acceptor emission in the sample with no enzyme. Fold change in the control sample thus is equal to 1. The QD-PDD reporter against BamHI demonstrated >20% fold change only in enzyme cocktails that contained BamHI and no non-specific reporting was observed (FIG. 3C). The EcoRI-reporting QD-PDD demonstrated >15% fold change, while the NcoI-QD-PDD showed 10% fold change. Regardless, all three QD-PDDs, at varying degrees of threshold fold change, could be activated in the presence of the corresponding enzyme only, thereby functioning in a multiplex manner.

QD-PDD Operation in a Cell-Free System

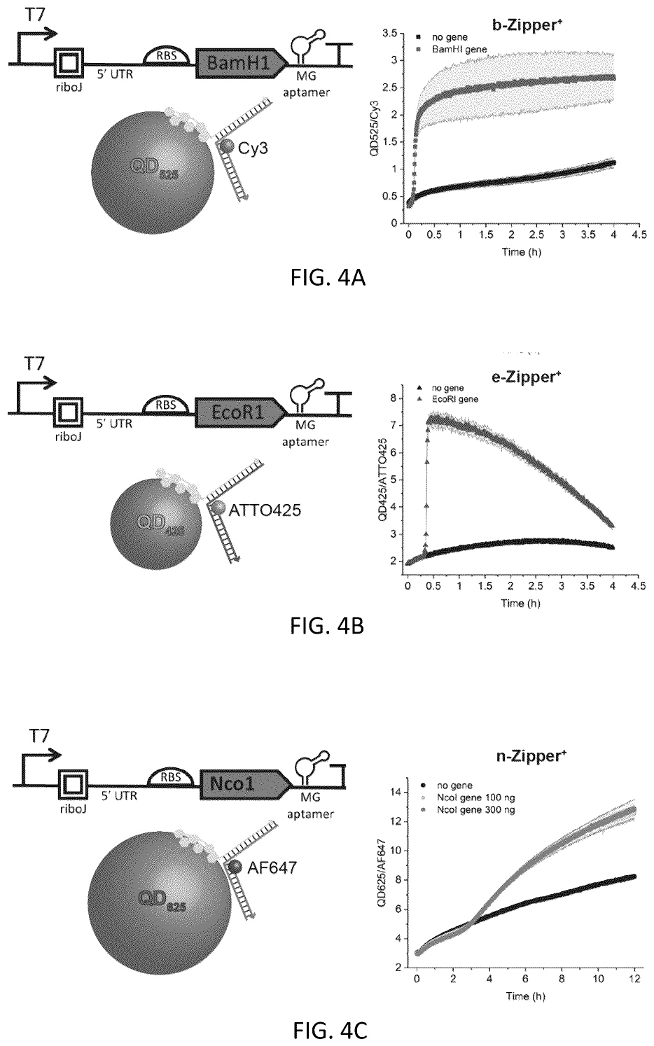

The operation of QD-PDDs in cell-free systems is such that expression of one or more of the three restriction enzymes could trigger the corresponding QD-PDD reporters in a triple-reporter mix (FIGS. 4A-4C). To that end, genes expressing BamHI, EcoRI, and NcoI were identified, obtained from commercial vendors (Appendix Table S2). The proteins were expressed under a T7 promoter using commercially available PURExpress cell-free system (NEB). As shown in the drawing figures, the constructs included the RiboJ genetic insulator followed by a 5′ untranslated region (UTR) and the ribosyme binding site (RBS). To monitor protein (restriction enzyme) synthesis in the cell-free system, the sequences encoding for the three restriction enzymes were each designed with a downstream malachite green aptamer (MG aptamer). First the expression of each protein via cell-free extract in the presence of their corresponding QD-PDD was individually tested for compatibility (while not monitoring QD-PDD functionality). All three restriction enzymes transcribed via T7 RNA polymerase (RNAP) led to an increase in malachite green fluorescence with time in the presence of the corresponding DNA (Appendix Figure S5A). Checking the extent of compatibility in reverse, each of the QD-PDD reporters was introduced in the cell-free system containing the corresponding DNA such that protein synthesis was expected to recognize and cleave the dsDNA restriction site on the QD-PDD and trigger a FRET change in QD-PDD fluorescence. These data show that the QD-PDDs remained functionality active with varying levels of performance. Time point t=0 h represents the initiation of PURExpress protein synthesis from 100 ng DNA supplied. Individually, b-Zipper⁺ and e-Zipper⁺ performed rapidly with maximum QD/acceptor ratio achieved in less than 1 h (FIGS. 4A and 4B). On the other hand, both configurations of NcoI-based reporters showed delayed response to the PURExpress-synthesized enzyme in >4 h (FIG. 4C), even at higher quantities of input gene (100 ng and a higher DNA dosage of 300 ng), as shown in Appendix Figure S5B.

QD-PDD Multiplexing in a Cell-Free System

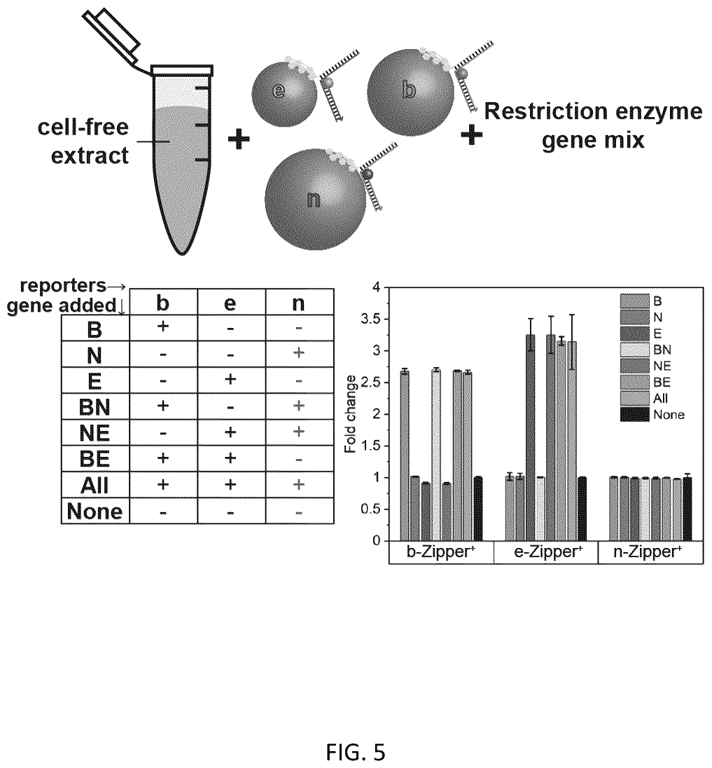

Analogous to triple-reporter assay of FIG. 3C, eight combinations of the DNA expressing the three enzymes were given as input to the triple-reporter in PURExpress to test their multiplexing efficacy (FIG. 5 and Appendix Figure S6). The b- and e-reporters performed predictably with superior fold change and no false positives as compared to their performance discussed above against pre-made enzymes (as shown in FIG. 3C), with 150% fold change in the b-reporter and over 300% fold change in the e-reporter. The n-reporter was inactive in the presence of all gene input combinations. It is possible that the 625 nm emitting QD used for NcoI QD-PDDs could have poor compatibility with the cell-free system, or the MGapt production is affecting n-reporter functionality. Nevertheless, for proof-of-principle, the b- and e-reporters demonstrated successful multiplexing even in the presence of the inactive n-reporter.

### Further Embodiments

Although the term “dye strand” is used here and the examples employed dyes as FRET partners, the use of other forms of quenchers in the dye strand is contemplated. These can include standard dark quenching dyes such as QSY 7, QSY9, or BHQ-10; which can be regarded as non-emissive acceptor dyes with broad absorption spectra. Other suitable quenchers can include metal complexes or metal nanoparticles which have also been shown to quench QDs. For example, see Nano Lett. 2007, 7, 10, 3157-3164; ACS Nano 2013, 7, 10, 9489-9505; ACS Nano 2012, 6, 6, 5330-5347; and Nature Materials volume 2006, vol. 5, pages 581-589.

Advantages:

Utilizing the described QD-based assemblies as reporters in cell-free extracts for multiplexed biosensing assays offers several inherent advantages over other (bio)reporters:
(1) Use of QDs as donors in FRET-based assays bring with it a host of inherent advantages that are cumulatively unavailable to any other fluorophore in the same role, these include;
   (i) The ability to tune the QD emission to better match an acceptor absorption profile (i.e., an acceptor absorption minima) while minimizing direct excitation of the acceptor.
   (ii) The ability to array multiple acceptors around a central QD which allows tuning of the FRET efficiency by proportionally increasing the acceptor absorption cross-section, this allows for a choice of an initial FRET efficiency that provides the most dynamic changes during a subsequent sensing/reporting event.
   (iii) The ability to excite multiple QDs at a single wavelength that is distinct and significantly blue shifted from their emission. Here, each QD can be paired with its own distinct acceptor to provide for multiplexing where the number of distinct reporters is only limited by the ability to resolve the FRET changes.
   (iv) The ability to use multiphoton excitation or to use a further long-lifetime donor to the QD to minimize interference and background in cellular extracts.
   (v) The ability to use the QD surface directly to controllably attach multiple sensing or acceptor moieties in contrast to other organic-fluorophore based donors.
(2) Components of the cell-free extract, especially if the proteins display a terminal (His)₆ moiety that was used in their original purification, can also assemble to the QD surface and act to enhance the performance of the reporter by channeling like effects between the enzymes.
(3) The same peptide PNA in combination with orthogonal QD-dye FRET pairs and the requisite DNA strand can be assembled to create a library of QD-PDD bioreporters against different analytes.
(4) QDs have similar absorbance properties, which enables simultaneous excitation of multiple QDs. A cocktail of QD-PDD reporters can be created without the worry of crosstalk or leakage and simultaneously activated with a single excitation wavelength.
(5) Assembly of the QD-PDD reporter is rapid and requires just minutes.
(6) No need for any covalent chemistry as the reporter is completely self-assembled.
(7) Modularity—any given QD reporter construct can be easily reconfigured to be cleaved by another restriction enzyme simply by changing the sequence which that enzyme recognizes.
(8) The reporter design can incorporate other nucleic acids such as RNA.
(9) The design inherently lends itself to incorporation of other hydrolytic or ‘cleavage’ enzymes such as proteases or lipases by reconfiguring the recognition and or cleaved portion of the reporter to the appropriate material. This can increase the bioorthogonality and applicability of both the reporter and the sensor it is used with.
(10) The ability to convert the observed change in FRET for a given reporter to quantitative units of enzyme velocity through the use of an appropriate FRET calibration curve.
(11) Increased sensing efficiency through enzyme processivity around the QD reporter rather than multiple cleavage events on independent targets which have the enzyme diffusing away between each cleavage event.
(12) The ability to put 2 different reporters with 2 different acceptor dyes on the same QD donor and still access similar function.
(13) Access to concentric multiFRET process and the concomitant orthogonal multisensing processes inherent to that.
(14) The ability to function with other types of FRET acceptors including dark quenching dyes or even fluorescent proteins when appropriately conjugated and incorporated into the reporter.
(15) The ability to access other energy transfer modalities for reporting such as electron transfer with the QD by replacing the acceptor fluorophore with an appropriate redox active electron donor or acceptor such as an electroactive ruthenium phenanthroline complex, for example.
(16) The ability to tether or attach or capture the QD sensing portion on the surface of a microtiter well plate or other surface (e.g., a microfluidics device) for the reporting.
(17) The ability to lyophilize or freeze the reporters alone or with cell extracts and reconstitute or defrost when needed.
(18) The ability to work in a paper-based format.
(19) The ability to sense 2 different types of enzyme function such as a protease and a restriction enzyme.
(20) The QD can also function as an acceptor with the appropriate donors attached to the DNA such as long-lifetime Tb chelates.
(21) The QDs and, in turn, the reporter configuration can be excited and driven in ‘un-powered’ modalities for field use by incorporating in BRET or CRET driven chemical sensitization.
(22) There is no need for a purification step after assembly.

### CONCLUDING REMARKS

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

### REFERENCES

Peptide Nucleic Acids
1. Pellestor, F., Paulasova, P. The peptide nucleic acids (PNAs), powerful tools for molecular genetics and cytogenetics. Eur Hum Genet 12, 694-700 (2004).

Quantum Dots and FRET
1. Medintz, I. L. and H. Mattoussi. Quantum dot-based resonance energy transfer and its growing application in biology. Phys. Chem. Chem. Phys. 11, 17-45 (2009).
2. Medintz, I. L., Clapp, A. R., Mattoussi, H., Goldman, E. R., Fisher, B., Mauro, J. M. Self-assembled nanoscale biosensors based on quantum dot FRET donors. Nature Materials 2:630-638 (2003).
3. Algar, W. R., Wegner, D., Huston, A., Blanco-Canosa, J. B., Stewart, M. H., Armstrong, A., Dawson, P. E., Hildebrandt, N. and Medintz, I. L. Quantum Dots as Simultaneous Acceptors and Donors in Time-Gated Forster Resonance Energy Transfer Relays: Characterization and Biosensing. Journal of the American Chemical Society 134, 1876-1891
   (2012).
4. Spillmann, C. M; Ancona, M. G.; Buckhout-White, S.; Algar, W. R.; Stewart, M. H.; Susumu, K.; Huston, A. L.; Goldman, E. R.; Medintz, I. L. Achieving Effective Terminal Exciton Delivery in Quantum Dot Antenna-Sensitized Multistep DNA Photonic Wires. ACS Nano 7, 7101-7118 (2013).
5. Algar, W. R.; Kim, H.; Medintz, I. L.; Hildebrandt, N. Emerging Non-Traditional Forster Resonance Energy Transfer Configurations with Semiconductor Quantum Dots: Investigations and Applications. Coordination Chemistry Reviews 263-264, 65-85 (2014).
6. Hildebrandt, N., Spillmann, C. M., Algar, W. R., Pons, T., Stewart, M. H., Oh, E., Susumu, K., Diaz, S. A., Delehanty, J. B., Medintz, I. L. Energy Transfer with Semiconductor Quantum Dot Bioconjugates: A Versatile Platform for Biosensing, Energy Harvesting, and Other Developing Applications. Chemical Reviews 117, 536-711 (2017).

Quantum Dots and Multiplexing
1. Clapp, A. R., Medintz, I. L., Fisher, B. R., Uyeda, H. T., Goldman, E. R., Bawendi, M. G., Mattoussi, H. Quantum Dot-Based Multiplexed Fluorescence Resonance Energy Transfer. J. Am. Chem Soc. 127, 18212-18221 (2005).
2. Goldman, E. R., Clapp, A. R., Anderson, G. P., Mauro, J. M., Uyeda, H. T., Medintz, I. L., Mattoussi, H. Multiplexed Toxin Analysis Using Four Colors of Quantum Dot Fluororeagents. Analytical Chemistry 76:684-688 (2004).
3. Medintz, I. L., Farrell, D., Susumu, K., Trammell, S., Deschamps, J. R., Brunel, F., Dawson, P., and Mattoussi, H. Multiplex charge transfer interactions between quantum dots and peptide-bridged ruthenium complexes. Analytical Chemistry. 81, 4831-4839 (2009).
4. Sapsford. K., Spindel, S., Jennings, T. L., Triulzi, R. C., Tao, G., Algar, W. R., Medintz, I. L. Optimizing two-color semiconductor nanocrystal immunoassays in single well microtiter plate formats. Sensors 11, 7879-7891 (2011).
5. Algar, W. R Ancona, M. G.; Malanoski, A. P.; Susumu, K.; and Medintz, I. L. Assembly of a Concentric Forster Resonance Energy Transfer Relay on a Quantum Dot Scaffold: Characterization and Application to Multiplexed Protease Sensing. ACS Nano 6, 11044-11058 (2012).

Quantum Dots and Biosensing
1. Medintz, I. L., Clapp, A. R., Brunel, E M., Tiefenbrunn, T., Uyeda, H. T., Chang, E. L., Deschamps, J. R., Dawson, P. E. and H. Mattoussi. Proteolytic activity monitored by FRET through quantum-dot peptide conjugates. Nature Materials 5, 581-589 (2006).
2. Boeneman, K., Mei, B., Dennis, A., Bao, G., Deschamps, J. R., Mattoussi, H. Medintz, I. L. Sensing Caspase 3 activity with quantum dot-fluorescent protein assemblies. Journal of the American Chemical Society. 131, 3828-3829 (2009).
3. Prasuhn, D. E., Feltz, A., Blanco-Canosa, J. B., Susumu, K., Stewart, M. H., Mei, B. C., Yakovlev, A., Loukov, C., Mallet, J. M., Oheim, M., Dawson, P. E. and Medintz, I. L. Quantum dot peptide biosensors for monitoring caspase 3 proteolysis and calcium ions. ACS Nano 4, 5487-5497 (2010).
4. Algar, W. R., Malanoski, A., Deschamps, J. R., Blanco-Canosa, J. B., Susumu, K., Stewart, M. H., Johnson, B. J., Dawson, P. E. and Medintz, I. L. Proteolytic Activity at Quantum Dot-Conjugates: Kinetic Analysis Reveals Enhanced Enzyme Activity and Localized Interfacial “Hopping”. Nano Letters 12, 3793-3802 (2012).
5. Diaz, S. A., Malanoski, A., Susumu, K., Hofele, R. V., Oh, E., Medintz, I. L. Probing the Kinetics of Quantum Dot-Based Proteolytic Sensors. Analytical and Bioanalytical Chemistry 407, 7307-7318 (2015).
6. Diaz, S. A., Breger, J. C., Medintz, I. L. Monitoring Enzymatic Proteolysis Using Either Enzyme- or Substrate Bioconjugated Quantum Dots. Methods in Enzymology 571, 19-54 (2016).
7. Diaz, S. A.; Gillanders, F.; Susumu, K.; Oh, E.; Medintz, I. L.; Jovin, T. M., Water-Soluble, Thermostable, Photomodulated Color-Switching Quantum Dots. Chemistry 2017, 23 (2), 263-267

Coordination of (Poly)Histidine Peptides to QDs
1. Sapsford, K. E., Pons, T., Medintz, I. L., Higashiya, S., Brunel, E M., Dawson, P. E. and H. Mattoussi. Kinetics of metal-affinity driven self-assembly between proteins or peptides and CdSe—ZnS quantum dots. J. Physical Chem. C. 111, 11528-11538 (2007).
2. Prasuhn, D. E., Blanco-Canosa, J. B., Vora, G. J., Delehanty, J. B., Susumu, K., Mei, B. C., Dawson, P. E. and Medintz, I. L. Combining chemoselective ligation with polyhistidine-driven self-assembly for the modular display of biomolecules on quantum dots. ACS Nano 4, 267-278 (2010).
3. Dennis, A. M. Sotto, D. Mei, B. C., Medintz, I. L. Mattoussi, H. and Bao, G. Surface ligand effects on metal-affinity coordination to quantum dots: Implications for nanoprobe self-assembly. Bioconjugate Chemistry 21, 1160-1170 (2010).
4. Boeneman, K., Deschamps, J., Buckhout-White, S., Prasuhn, D., Blanco-Canosa, J., Dawson, P., Stewart, M., Susumu, K., Goldman, E. R., Ancona, M., Medintz, I. L. Quantum dot DNA bioconjugates: attachment chemistry strongly influences the resulting composite architecture. ACS Nano 4, 7253-7266 (2010).
5. Boeneman, K., Prasuhn, D., Blanco-Canosa, J., Dawson, P., Melinger, J. S., Ancona, M., Stewart, M., Susumu, K., Huston, A., Medintz, I. L. Self-assembled quantum dot-sensitized multivalent photonic wires. Journal of the American Chemical Society 132, 18177-18190 (2010).
6. Blanco-Canosa, J.; Wu, M.; Susumu, K.; Petryayeva, E.; Jennings, T. L.; Dawson, P. E.; Algar, W. R.; Medintz, I. L. Recent Progress in the Bioconjugation of Quantum Dots. Coordination Chemistry Reviews 263-264, 101-137 (2014).

Cell-Free Transcription/Translation Systems
1. Moore, S. J., MacDonald, J. T., and Freemont, P. S. Cell-free synthetic biology for in vitro prototype engineering. Biochem. Soc. Trans. 45, 785-791, (2017). DOI: 10.1042/BST20170011
2. Sun, Z. Z., Yeung, E., Hayes, C. A., Noireaux, V., and Murray, R. M. Linear DNA for rapid prototyping of synthetic biological circuits in an Escherichia coli based TX-TL cell-free system. ACS Synth. Biol. 3 (6), 387-97, (2014).
3. Schinn, S. M., Broadbent, A., Bradley, W. T., and Bundy, B. C. Protein synthesis directly from PCR: Progress and applications of cell-free protein synthesis with linear DNA. New Biotechnol. 33, 480-487, (2016). DOI: 10.1016/j.nbt.2016.04.002
4. Kay, J. E. and Jewett, M. C. A cell-free system for production of 2,3-butanediol is robust to growth-toxic compounds. Metab Eng. Commun. 10, e00114, (2020). DOI: 10.1016/j.mec.2019.e00114

Biosensing with Cell Free Transcription Translation Systems
1. Silverman, A. D., Karim, A. S., and Jewett, M. C. Cell-free gene expression: an expanded repertoire of applications. Nat. Rev. Genet. 21 (3), 151-170, (2020). DOI: 10.1038/s41576-019-0186-3
2. Pardee, K., Green, A. A., Ferrante, T., Cameron, D. E., Daleykeyser, A., Yin, P., and Collins, J. J. Paper-based synthetic gene networks. Cell 159, 940-954, (2014). DOI: 10.1016/j.cell.2014.10.004
3. Pardee, K., Green, A. A., Takahashi, M. K., Braff, D., Lambert, G., Lee, J. W., Ferrante, T., Ma, D., Donghia, N., Fan, M., Daringer, N. M., Bosch, I., Dudley, D. M., O'Connor, D. H., Gehrke, L., and Collins, J. J. Rapid, Low-Cost Detection of Zika Virus Using Programmable Biomolecular Components. Cell 165 (5), 1255-1266, (2016). DOI: 10.1016/j.cell.2016.04.059
4. McNerney, M. P., Zhang, Y., Steppe, P., Silverman, A. D., Jewett, M. C., and Styczynski, M. P. Point-of-care biomarker quantification enabled by sample-specific calibration. Sci. Adv. 5 (9), eaax4473, (2019). DOI: 10.1126/sciadv.aax4473
5. Sadat Mousavi, P., Smith, S. J., Chen, J. B., Karlikow, M., Tinafar, A., Robinson, C., Liu, W., Ma, D., Green, A. A., Kelley, S. O., and Pardee, K. A multiplexed, electrochemical interface for gene-circuit-based sensors. Nat. Chem. 12 (1), 48-55, (2020). DOI: 10.1038/s41557-019-0366-y

What is claimed is:

1. A quantum dot reporter comprising:
a quantum dot (QD);
a peptide-PNA strand comprising (1) a peptide nucleic acid (PNA) comprising a PNA sequence and (2) a peptide comprising a polyhisitine sequence effective to bind the peptide-PNA strand to the QD;
a nucleic acid bridge strand comprising a sequence complementary the PNA sequence and configured to bind the bridge strand to the peptide-PNA via hybridization, a target sequence for a restriction endonuclease enzyme of interest, and a bridge terminal spacer; and
a nucleic acid quencher strand comprising a sequence complementary to the target sequence thus binding the quencher strand to the bridge strand, a second terminal spacer, and quencher configured as a Forster resonance energy transfer (FRET) partner of the QD, such that the quencher is positioned to quench QD fluorescence bound to the bridge strand,
wherein cleavage by the restriction endonuclease enzyme of interest causes separation of the quencher from the QD and an increase of QD fluorescence.

2. The reporter of claim 1, wherein said quencher is a fluorescent dye.

3. A system comprising two or more different reporters according to claim 1, each of which having distinct quantum dots and target sequences for different restriction endonucleases of interest.

4. A method of monitoring a cell-free reaction comprising:
conducting a cell-free reaction effective to produce a restriction endonuclease enzyme of interest; and
introducing a quantum dot reporter and monitoring fluorescence thereof as an indication of reaction progress,
wherein the quantum dot reporter comprises a quantum dot (QD); a peptide-PNA strand comprising (1) a peptide nucleic acid (PNA) comprising a PNA sequence and (2) a peptide comprising a polyhisitine sequence effective to bind the peptide-PNA strand to the QD; a nucleic acid bridge strand comprising a sequence complementary the PNA sequence and configured to bind the bridge strand to the peptide-PNA via hybridization, a target sequence for the restriction endonuclease enzyme of interest, and a bridge terminal spacer; and a nucleic acid quencher strand comprising a sequence complementary to the target sequence thus binding the quencher strand to the bridge strand, a second terminal spacer, and quencher configured as a Forster resonance energy transfer (FRET) partner of the QD, such that the quencher is positioned to quench QD fluorescence bound to the bridge strand, wherein cleavage by the restriction endonuclease enzyme of interest causes separation of the quencher from the QD and an increase of QD fluorescence.

5. The method of claim 4, wherein the monitoring is in multiplex of multiple distinct reactions running concurrently via the system of claim 3.
